(19) United States
(12) Patent Application Publication
(10) Pub. No.: US 2006/0229256 A1
(43) Pub. Date: Oct. 12, 2006

(54) Enternal administration of arginine and glutamine for abnormal vascular proliferation

(75) Inventors: Joshua C. Anthony, Evansville, IN (US); Josef Neu, Gainesville, FL (US); Maria B. Grant, Fairfield, FL (US)

Correspondence Address: NELSON MULLINS RILEY & SCARBOROUGH, LLP, MERIDIAN, SUITE 1700, 1320 MAIN STREET, COLUMBIA, SC, 29201, US

(73) Assignees: Bristol-Myers Squibb Company, Princeton, NJ (US); University of Florida Research Foundation, Incorporated, Gainesville, FL (US)

(21) Appl. No.: 11/392,913

(22) Filed: Mar. 29, 2006

Related U.S. Application Data
(63) Continuation-in-part of application No. 10/950,734, filed on Sep. 27, 2004, now abandoned.
(60) Provisional application No. 60/506,413, filed on Sep. 26, 2003.

(51) Int. Cl. A61K 38/04; A61K 31/198

(52) U.S. Cl. 514/19; 514/563; 514/565

(57) ABSTRACT

The subject invention provides a method and compositions for preventing and/or treating abnormal vascular proliferation in a human infant where the method involves enterally administering arginine and glutamine to the infant in about equimolar amounts to provide a total amount of arginine and glutamine that is effective to prevent or treat the abnormal vascular proliferation. Arginyl-glutamine dipeptide was shown to be an advantageous form in which to provide the two amino acids.

### CROSS REFERENCE TO RELATED PATENTS AND PATENT APPLICATIONS

The subject matter of the present invention is related to copending U.S. patent application Ser. No. 10/951,150, filed Sep. 27, 2004, and is a Continuation-in-part of U.S. patent application Ser. No. 10/950,734, filed Sep. 27, 2004, which was a non-provisional of U.S. Provisional Patent Application No. 60/506,413, filed Sep. 26, 2003, and each of which is incorporated herein by reference in its entirety.

### BACKGROUND OF THE INVENTION

(1) Field of the Invention

The present invention relates to the prevention and/or treatment of abnormal (pathological) vascular proliferation, and more particularly to the prevention and/or treatment of pathological vascular proliferation by enteral administration of arginine and glutamine and, in particular, arginyl-glutamine dipeptide.

(2) Description of the Related Art

People suffering from visual impairment face many challenges in performing routine daily activities and/or may not be able to fully enjoy the visual aspects of their surroundings. Of particular concern with regard to the current invention are visual impairments caused by damage to the retina, which occur in conditions such as diabetic retinopathy and retinopathy of prematurity.

Diabetic retinopathy is a progressive disease characterized by abnormalities of the blood vessels of the retina caused by diabetes, such as weakening of the blood vessel walls, leakage from the blood vessels, and bleeding and scarring around new vessels. Diabetic retinopathy results in impairment of a person's vision causing severely blurred vision and, potentially, blindness.

Diabetes affects over 16 million Americans. The World Health Organization indicates that diabetes afflicts 120 million people worldwide, and estimates that this number will increase to 300 million by the year 2025. Diabetics are faced with numerous complications including kidney failure, non-traumatic amputations, an increase in the incidence of heart attack or stroke, nerve damage, and loss of vision. Diabetic retinopathy is a form of visual impairment often suffered by diabetics.

Due to significant medical advancements, diabetics are able to live much longer than in the past. However, the longer a person has diabetes the greater the chances of developing diabetic retinopathy. Affecting over 5.3 million Americans, diabetic retinopathy is the leading cause of blindness among adults in the United States. Annually, in the United States, between 12,000 and 24,000 people lose their sight because of diabetes.

While management of diabetic retinopathy has improved, risk of complications, such as loss of visual acuity, loss of night vision and loss of peripheral vision, remains significant and treatment sometimes fails. Currently, laser photocoagulation is the most effective form of therapy for advanced disease. Unfortunately, current treatment options are inadequate and the disease is often progressive even with successful glucose control.

Retinopathy of prematurity (ROP) is a disorder of retinal blood vessel development in the premature infant. Under normal development, blood vessels grow from the back central part of the eye out toward the edges. In premature babies, this process is not complete and the abnormal growth of the vessels proliferate leading to scar tissue development, retinal detachment and possibly complete blindness.

ROP is the major cause of blindness in children under the age of 7. The salient pathological features are neovascularization in the retinal vascular endothelium with edema and breakdown in the blood-retinal barrier (BRB) that leads to hemorrhage, tissue damage and retinal scarring ultimately leads, in the severest cases, to blindness.

Improved care in the neonatal intensive care unit has reduced the incidence of retinopathy of prematurity in moderately premature infants. Ironically, however, increasing rates of survival of very premature infants, who would have had little chance of survival in the past, has increased the occurrence of retinopathy of prematurity. Since these very premature infants are at the highest risk of developing ROP, it is of great concern that the condition may actually be becoming more prevalent again.

For those babies in whom retinopathy progresses, treatment is necessary. Cryotherapy and laser treatment have some effect in advanced stages of the disease, saving a degree of vision in a proportion of the eyes that would otherwise have been blinded, but prevention awaits a better understanding of major causative factors and underlying pathophysiology.

Current research shows promise that the prevention of retinal blood vessel damage, which marks retinopathy, may be achieved by the utilization of certain compounds. It has been demonstrated that, in retinal epithelial cells, glutamine deprivation can lead to upregulation of vascular endothelial growth factor (VEGF) expression (Abcouwer S. et al., “Response of VEGF expression to amino acid deprivation and inducers of endoplasmic reticulum stress”, Invest Ophthalmol Vis Sci, August 2002, pp. 2791-8, Vol. 43, No. 8). Most sick premature infants are deprived of glutamine during the time they receive supplemental oxygen, a known predisposing factor in the development of ROP. The over expression of VEGF during this time period is also thought to be involved in the pathogenesis of ROP providing glutamine supplements during this time period could potentially down-regulate VEGF. Arginine is substrate for the reaction that produces nitric oxide, a very potent vasodilator, vasodilation in retinal blood vessels also prevents neovascularization. Nitric oxide also has numerous other beneficial effects and is now commonly used for treatment of lung disease in critically ill infants.

It is well known that proteins are converted to amino acids in the digestive system and that the resulting amino acids are used by the body for growth and development. Proteins and peptides administered for therapeutic or preventative measures are also well-known. Oligopeptides are better absorbed in the intestines than individual amino acids.

European Patent Application No. 0,182,356 discloses a nutritional composition containing at least one oligopeptide consisting of a dipeptide or a tripeptide wherein the N-terminal amino acid residue is selected from the class consisting of alanine, lysine and arginine.

One group conducting research in this area concluded that glycine is generally superior to other amino acids as the N-terminal amino acid residue in a dipeptide. This superiority was attributed to a greater fraction of such an intravenously administered dipeptide reaches the tissues. S. Adibi et al., Influence of Molecular Structure on Half-life and Hydrolysis of Dipeptides in Plasma: Importance of Glycine as N-Terminal Amino Acid Residue, 35 Metabolism 850, 835 (1986).

Two European patents, 0,087,751 and 0,087,750 disclose water-soluble peptides. The '751 patent discloses a method to parenterally administer low water-soluble amino acids. Two amino acids, tyrosine and cystine, individually have low solubility in water. These amino acids, however, are clinically useful and, therefore, it was desirable to find an effective formulation. The '751 patent describes an infusion method which involves bonding these relatively insoluble amino acids to the amino acid lysine to produce a tripeptide.

The '750 patent discloses the infusion of glutamine as a derivative substituted by α-aminoacyl residues on the α amino group. That is, glutamine is in the “c-terminal” position, in that its alpha amino nitrogen becomes part of the peptide bond with the other amino acid. The preferred dipeptide preparation disclosed in the '750 patent is alanyl-glutamine. The aminoacylation of glutamine is reported to achieve a stabilization of the terminal amide group.

Experiments involving the use of total parenteral nutrition (TPN) containing glycyl-glutamine dipeptides, however, suggest potential adverse effects of the TPN formulation containing glycyl-glutamine (U.S. Pat. No. 5,189,016).

Recently, the use of an arginyl-glutamine dipeptide for the prevention of muscle breakdown and microbial infections has recently been described. See, WO 03/017787. These amino acids have also been described in complex compositions (Miyazawa et al. (1976) Journal of Faculty of Fisheries and Animal Husbandry Hiroshima 15(2):161-169; and JP 2119762).

Two commercially available dipeptides of glutamine are Dipeptiven, which is an alanyl-glutamine (Fresenius Laboratories, Germany) and Glamin (Pharmacia and Upjohn Laboratory, Sweden), which is an amino acid solution containing glycyl-glutamine dipeptide. High-protein sports drinks containing arginine, glutamine and other amino acids are available and Amino Vital®, Ajinomoto Co., Inc., is one example.

Neu, in U.S. Patent Application Publication US 2004/0097426, described compositions containing an arginyl-glutamine dipeptide and the use of these compositions for prevention of muscle breakdown and microbial infection.

Later, in U.S. Patent Application Publication No. US 2005/0070484, Neu and Grant described the application of arginyl-glutamine dipeptides in preventing and/or treating pathological proliferation of blood vessels, and demonstrated the efficacy of the intraperitoneal administration of arginyl-glutamine dipeptide for decreasing retinopathy in a mouse model of retinopathy of prematurity.

Neu et al., in U.S. Patent Application Publication US 2005/0089547, further described the inclusion and use of arginyl-dipeptide in infant formula and dietary supplements for the prevention and/or treatment of the pathological proliferation of blood vessels, and in particular, for the prevention or treatment of retinopathy of prematurity, diabetic retinopathy, vascular proliferative retinopathy, or proliferation of abnormal vascularization.

With the increase of adult onset diabetes, longer life span for diabetics and high rate of survival of very premature infants, many individuals are now at even greater risk for developing retinopathy. Although treatment options, such as laser therapy, exist for both conditions, the results are inadequate and the disease often remains progressive. There remains a great need in the art for compositions and methods which prevent and/or treat retinal diseases. Moreover, there is a need for compositions and methods that can easily be administered to infants in forms and manners that are readily accepted by the infant and the care-giver.

### SUMMARY OF THE INVENTION

Briefly, therefore the present invention is directed to a novel method for preventing and/or treating abnormal vascular proliferation in a subject, the method comprising enterally administering arginine and glutamine to the subject in about equimolar amounts to provide a total amount of arginine and glutamine that is effective to prevent or treat the abnormal vascular proliferation.

The present invention is also directed to a novel infant formula comprising arginine and glutamine in about equimolar amounts and in a total amount to provide the combination of arginine and glutamine to a human infant in an amount of from about 100 mg/kg·day to about 1000 mg/kg·day.

The present invention is also directed to a novel infant nutritional supplement comprising amino acids consisting essentially of arginine and glutamine in about equimolar amounts.

Among the several advantages found to be achieved by the present invention, therefore, may be noted the provision of compositions and methods which prevent and/or treat retinal diseases, and the provision of compositions and methods that treat and prevent pathological vascular proliferation and which can easily be administered to infants in forms and manners that are readily accepted by the infant and the care-giver.

### BRIEF DESCRIPTION OF THE DRAWINGS

FIG. 1 is a bar chart showing the effect of the administration of Arg-gln dipeptide on VEGF secretion in human RPE cells as a function of the level of Arg-gln, and indicates that exposure to the dipeptide resulted in a statistically significant dose-dependent decrease in VEGF expression;

FIG. 2 is a bar chart showing the dose effect of the Arg-gln dipeptide on retinal vascular proliferation in mice and indicated significant reduction in preretinal nuclei at even the lowest dose tested (1 gm/kg·day), and an 80% reduction at the highest dose tested (5 gm/kg·day);

FIG. 3 is a bar chart showing the effect of the parenteral administration of Arg-gln dipeptide on VEGF mRNA in the eyes of neonatal mice exposed to hypoxia as a function of time, and indicates that the increase in VEGF mRNA was much lower in pups receiving the dipeptide than in those receiving the vehicle; and

FIG. 4 is a bar chart showing the effect of administering Arg-gln dipeptide by gavage in reducing the average nuclei per section in the eyes of neonatal mice exposed to hyperoxia to induce retinal angiogenesis and to mimic retinopathy of prematurity at dosage levels of zero (vehicle), 1 gm/kg·day, 2.5 gm/kg·day, and 5 gm/kg·day.

### DETAILED DESCRIPTION OF THE PREFERRED EMBODIMENTS

In accordance with the present invention, it has been discovered that enteral administration of effective amounts of arginine and glutamine in approximately eqimolar ratio to neonatal animals, and in particular to human infants, is an effective method to prevent and/or treat the proliferation of abnormal blood vessels. In particular, the subject invention provides a novel, safe and affordable therapy for the prevention or the treatment of pathological ocular vascular proliferation. In a particularly effective embodiment, the inventors have shown that enteral administration of certain amounts of arginine and glutamine in the form of a dipeptide was remarkably effective for reducing the proliferation of retinal blood vessels in a mouse model for retinopathy of prematurity.

The subject invention contemplates the enteral administration of roughly equimolar amounts of arginine and glutamine in any form that can be ingested and absorbed by an infant. Arginine and glutamine as free amino acids, or as salts, prodrugs, or precursors of such amino acids can be used. Also, an arginyl-glutamine dipeptide itself may be administered, as well as oligopeptides, peptides, proteins, protein hydrolyzates, and any other materials that could serve as a source of roughly equimolar amounts of arginine and glutamine or of the arginyl-glutamine dipeptide. Examples include peptides of polyalanine and polyglutamine, peptides containing blocks of polyalanine and polyglutamine, and peptides of alternating alanine and glutamine.

In the case of oligopeptides, peptides, and proteins that contain the arginyl-glutamine dipeptide, these prodrug formulations may be designed with, for example, cleavage sites adjacent to each side of the arginyl-glutamine dipeptide so that the dipeptide is generated upon exposure to enzymes, acids or other factors. In one embodiment, a polypeptide can be prepared with multiple arginyl-glutamine dipeptides separated by cleavage sites, thereby creating multiple dipeptides upon exposure to the cleaving factor. This cleaving to create the dipeptide can be done as part of a production process or in vivo as the result of, for example, digestive enzymes and/or acids.

As contemplated in the subject invention, where the Arg-gln dipeptide is provided by a prodrug, the prodrug can be converted to a biologically active compound at a controlled rate via passive (such as by aqueous hydrolysis) or biologically mediated (such as biocatalytic or enzymatic) mechanisms. An advantage of the in vivo conversion of the prodrug is that the ensuing dipeptide provides localized therapeutic effects in target disease tissue with high therapeutic margins of safety.

In one embodiment, it is useful to provide the arginine and glutamine in the form of arginyl-glutamine dipeptide. In this form, the subject invention provides the two amino acids in a dipeptide having excellent water solubility, stability to sterilization, long-term stability, and bioavailability for humans and animals. An advantage of the dipeptide in solution is its increased stability over free glutamine, for example, resulting in much lower cyclisation of glutamine into undesirable pyro-glutamate. In one form, the arginyl-glutamine dipeptide of the present invention has an N-terminal amino acid which is arginine and a C-terminal amino acid which is glutamine (referred to herein as Arg-gln). Furthermore, the use of the Arg-gln dipeptide provides equimolar amounts of each of the two amino acids.

The Arg-gln dipeptides of the subject invention can be readily synthesized and/or formulated by a person skilled in the art having the benefit of the instant disclosure. Alternatively, the dipeptides can be purchased commercially from, for example, Bachem Biosciences, Inc. which sells the H-Arg-Gln-OH salt. DIPEPTIVEN is available from Fresenius Kabi, Uppsala, Sweden, and is a 20% solution of N(2)-L-alanyl-glutamine. Further information is found in Fürst et al., The J. of Nutrition (Suppl): 2562S-2568S (2001). The Arg-gln dipeptides can be of any purity or grade, and can be of a purity and grade that is suitable for inclusion in the diet of human infants.

When it is said herein that arginine and glutamine are administered in roughly equimolar amounts, it is meant that the one mole of arginine is administered to an infant for roughly every mole of glutamine. As used herein, compositions having argine and glutamine in arginine:glutamine molar ratios of from about 1.2:1 to about 1:1.2 would be considered to contain roughly equimolar amounts of the two amino acids. Also, arginine:glutamine molar ratios of from about 1.15:1 to about 1:1.15, or from about 1.1:1 to about 1:1.1, or about 1:1, would also be considered to be roughly equimolar amounts. As mentioned above, the Arg-gln dipeptide provides an arginine:glutamine molar ratio of about 1:1.

It is envisioned that the supplementation of any source of arginine or any source of glutamine having an arginine:glutamine molar ratio that is outside the range of 1.2:1 to 1:1.2 with the amino acid that is deficient to bring the ratio to within that considered to be roughly equimolar, as that term is used herein, is also within the scope of the present invention.

The present invention is useful for the treatment or prevention of abnormal vascular proliferation in a subject. The subject in the present invention can be a mammal, and can be a human adult or a human infant. As used herein, the terms “human infant” refer to a human between birth and about 3 years of age. Human infants between birth and about 1 year of age are of particular interest, as are human infants that are born prematurely (premature infants).

In an embodiment of the present invention, the human infant is in need of treatment or prevention of abnormal vascular proliferation. An infant is in need of treatment of abnormal vascular proliferation if such pathology is present in the infant. For example, the presence of retinopathy of prematurity, vascular proliferative retinopathy, or proliferation of abnormal vascularization, identify an infant that is in need of treatment for abnormal vascular proliferation. An infant is in need of prevention of abnormal vascular proliferation if it is at risk for developing such pathology. Infants that are at risk for developing retinopathy of prematurity, vascular proliferative retinopathy, or proliferation of abnormal vascularization, are in need or prevention of abnormal vascular proliferation. Premature human infants, diabetic infants, and infants exposed to hypoxia or other conditions that are known to increase the risk of abnormal vascular proliferation are in need of prevention of abnormal vascular proliferation.

In an embodiment of the present invention, an adult human is in need of treatment or prevention of abnormal vascular proliferation. An adult is in need of treatment of abnormal vascular proliferation if such pathology is present. For example, the presence of retinopathy of prematurity, vascular proliferative retinopathy, diabetic retinopathy, or proliferation of abnormal vascularization, identify an adult human that is in need of treatment for abnormal vascular proliferation.

In a specific embodiment of the subject invention the arginyl-glutamine dipeptides described herein can be used for preventing the proliferation of abnormal retinal blood vessels in an infant. Thus, these dipeptides can be administered to premature infants or diabetics who are at risk for retinal disease.

In accordance with the teachings provided herein, aqueous compositions can be prepared that include arginine and glutamine in roughly equimolar amounts, or which contain at least one arginyl-glutamine dipeptide. The dipeptide can be added to enteral formulations, which can include nutritional supplements. As discussed in more detail below, in addition to the arginine and glutamine, or the Arg-gln dipeptides of the subject invention, the formulas, supplements, or nutritional solutions can contain, for example, carbohydrates, lipids, other amino acids, peptides, and/or proteins, vitamins, minerals and trace elements. The selection of the particular arginine/glutamine or Arg-gln dipeptide formulation depends upon the particular use. The administration of the Arg-gln dipeptide rather than free amino acids permits administration of the same amount of amino acid residue in solutions which are less hypertonic and therefore of lower osmolality.

In the present invention, the arginine and glutamine, or Arg-gln, is enterally administered to an infant. When it is said that the amino acids or dipeptide is administered enterally, it is referring to administration through the digestive tract. As used herein enteral administration includes oral feeding, intragastric feeding, transpyloric administration, or any other introduction into the digestive tract of the infant.

Enteral nutrition is cheaper and safer than TPN (Total Parenteral Nutrition) and is the preferred route when the integrity of the GI tract is preserved. When possible, oral administration is desirable, because it is the normal method for infant nutrition and one that is understood and accepted by the infant and persons providing nutrition to the infant.

Enteral administration of the arginine/glutamine or Arg-gln (the active agents) can be by any recognized method and can take place at any time. Formulations containing the active agents can be given once a day or multiple times per day. Administration of formulations containing the active agents can be alternated with administration of formulations that do not contain the active agents, or contain them at levels other than those that will provide arginine and glutamine to the infant in about eqimolar amounts to provide a total amount of arginine and glutamine of from about 100 mg/kg·day to about 1000 mg/kg·day.

The arginine/glutamine amino acids or Arg-gln dipeptide of the present invention can be enterally administered to an infant in any known and accepted form or manner. An embodiment of the present invention is a composition that can be an infant formula or a nutritional or dietary supplement.

The infant formula or nutritional supplement of the present invention can be milk-based, soy-based, or based on other food sources. The composition may be prepared as a powder or a liquid for formulas prepared for infant populations. The inventive composition may be prepared as a nutritionally complete diet by including necessary nutrients, including vitamins and minerals at acceptable levels. The subject composition can be in the form of a dietary product such as an infant formula, milk substitute, and meal replacement or supplement.

An embodiment of the invention is a nutritional or dietary supplement that contains arginine and glutamine in roughly equimolar amounts, or the Arg-gln dipeptide, or a precursor thereof (which may also be referred to herein as a prodrug). The dietary supplement is designed to be administered along with a food or nutritional composition, such as infant formula, and can either be intermixed with the food or nutritional composition prior to ingestion by the subject, or can be administered to the subject either before or after ingestion of a food or nutritional composition. The subject dietary supplement contains an amount of arginine and glutamine, or arginyl-glutamine dipeptide, or a precursor thereof, that is effective for the prevention or treatment of retinopathy of prematurity, diabetic retinopathy, vascular proliferative retinopathy, or proliferation of abnormal vascularization, and the like.

In an embodiment, the nutritional supplement comprises amino acids consisting essentially of arginine and glutamine in about equimolar amounts. In another embodiment, the arginine and glutamine are provided by the Arg-gln dipeptide.

The amount of the equimolar combination of arginine and glutamine, or the Arg-gln dipeptide or its salt or prodrug, that is an effective amount is an amount sufficient to evoke the desired pharmacological response. This is generally an amount sufficient to produce lessening of one or more of the effects of pathological vascular proliferation. In the case of retinopathy, it is an amount sufficient to produce regression of neovascularization and/or an amount sufficient to produce improved visual acuity.

In an embodiment of the present invention, the roughly equimolar combination of arginine and glutamine, or the Arg-gln dipeptide, is administered to an infant in an amount that is effective to treat and/or prevent abnormal vascular proliferation. This amount can be from about 0.001 to about 10,000 mg/kg·day (where the units of mg/kg·day refer to mg of the combination of arginine and glutamine in roughly equimolar amount, or mg of the Arg-gln dipeptide, per kg of infant body weight per day). The effective amount can also be from about 100 mg/kg·day to about 1000 mg/kg·day, or from about 200 mg/kg·day to about 800 mg/kg·day, or from about 250 mg/kg·day to about 600 mg/kg·day, or from about 300 mg/kg·day to about 600 mg/kg·day, or from about 300 mg/kg·day to about 500 mg/kg·day, or in an amount of about 500 mg/kg·day. Here, the amount of the active ingredients by weight refers to the amount of the equimolar combination of arginine and glutamine or the Arg-gln dipeptide, or the amount of their salts or precursors sufficient to provide the stated amount of amino acids or dipeptide.

In one embodiment, a novel infant formula containing roughly equimolar amounts of arginine and glutamine, or the arginyl-glutamine dipeptide, or precursor thereof, is nutritionally complete. By the term “nutritionally complete” is meant that the composition contains adequate nutrients to sustain healthy human life for extended periods. The infant formula of the invention contains ingredients which are designed to meet the nutritional needs of the human infant namely, a protein, carbohydrate and lipid source and other nutrients such as vitamins and minerals.

Besides the subject amino acids or dipeptide, the composition of the invention can contain an additional nitrogen source (i.e., amino acids and/or protein) in an amount to make the total amount of amino acids or protein to be typically about 1 g to about 10 g per 100 kcal of total composition, preferably about 2 g to about 6 g per 100 kcal; the amount of lipid source per 100 kcal of total composition is typically greater than 0 g up to about 6 g, preferably about 0.5 g to about 5.5 g and more preferably about 2 g to about 5.5 g; and the amount of non-fiber carbohydrate source per 100 kcal of total composition is typically about 5 g to about 20 g, preferably about 7.5 g to about 15 g. The amount of vitamins and minerals in the nutritionally complete composition is typically sufficient to meet 100% of the U.S. recommended daily intake (RDI) in about 500 to about 3,000 kcal, preferable is about 1,000 to about 3,000 kcal.

In one embodiment of the present nutritional composition the amount of vitamins and minerals is sufficient to meet 100% of the RDI in about 500 to about 3,000 kcal, preferably in about 1,000 to about 3,000 kcal. As used herein, the RDI's are intended to mean those published in the Federal Register, Vol. 58, No. 3, Wednesday, Jan. 6, 1993, page 2227 which are as follows: Vitamin A, 5,000 International Units; Vitamin C, 60 milligrams; Thiamin, 1.5 milligrams; Riboflavin, 1.7 milligrams; Niacin, 20 milligrams; Calcium, 1.0 gram; Iron, 18 milligrams; Vitamin D, 400 International Units; Vitamin E, 30 International Units; Vitamin B6, 2.0 milligrams; Folic acid, 0.4 milligrams; Vitamin B12, 6 micrograms; Phosphorus, 1.0 gram; Iodine, 150 micrograms; Magnesium, 400 milligrams; Zinc, 15 milligrams; Copper, 2 milligrams; Biotin, 0.3 milligram; Pantothenic acid, 10 milligrams.

In the present method, the subject infant formula or dietary supplement is administered to an infant in an amount that is sufficient to provide arginine and glutamine or the Arg-gln dipeptide in an amount effective to prevent or treat retinopathy of prematurity, diabetic retinopathy, vascular proliferative retinopathy, or proliferation of abnormal vascularization.

The effective dosage range can be determined by one skilled in the art having the benefit of the current disclosure. Naturally, such therapeutic dosage ranges will vary with the size, species and physical condition of the patient, the severity of the patient's medical condition, the particular dosage form employed, the route of administration and the like. In addition, a route of administration may be selected to slowly release the chemical, e.g., slow intravenous infusion.

It is well known that infants from birth to 36 months, for example, normally weigh from about 3.5 kg to about 14.2 kg. (See, e.g., Growth Charts, at http://www.cdc.gov′/growthcharts, 01/12/2006). It is also well known that infants that are fed nutritionally complete formula consume about 100 to about 200 ml/kg·day of the formula. Thureen, P. et al., Pediatr Res., 57 (5 Pt. 2): 95R-98R (2005), and Raiha, N. C. R. et al., Journal of Pediatric Gastroenterology and Nutrition, 35:275-281 (2002). The state of the art with regards to infant formula is predictable and well known, and the skill of those in the art is high. Therefore, in view of the dosage of the active agents that is taught above, the high skill level and high state of knowledge regarding formulation of infant formula, and the common knowledge in the field of infant weights and consumption amounts, it is well within the ability for the skilled practitioner to determine the effective amount of the arginine/glutamine or the Arg-gln dipeptide.

By way of example, for a newborn infant weighing 3.5 kg and consuming about 780 ml/day of liquid formula, the 780 ml of liquid infant formula would have to provide 1.75 gm of Arg-gln dipeptide in order to provide 500 mg/kg·day to the infant. This would require Arg-gln dipeptide at a concentration of about 2.24 gm/liter (wt./vol.) in the liquid formula. Moreover, if a powder was used to make up the liquid formula, then the Arg-gln dipeptide could be provided in the powder. Knowing that the normal level of reconstitution is about 15 g powder per 100 ml of liquid, a powder containing about 1.75 g of the Arg-gln dipeptide in the 117 g of powder for the 780 ml of formula needed for one day's feeding of the infant, or a powder with about 1.5% w/wt of the Arg-gln dipeptide would be required.

These calculations are easily repeated for any level of the dipeptide within the range taught by the specification, i.e. from 100 to 1000 mg/kg·day, and for an infant of any weight.

When an additional protein source is included in the subject infant formula, it can be non-fat milk solids, a combination of non-fat milk solids and whey protein, a partial hydrolysate of non-fat milk and/or whey solids, soy protein isolates, or partially hydrolyzed soy protein isolates. The infant formula can be casein predominant or whey predominant.

As used herein, the subject infant formula is not meant to include natural milk, or any other unmodified natural product, but rather refers to a formulation made by man in whole or in part by intermixing two or more ingredients.

The carbohydrate source in the infant formula can be any suitable carbohydrate known in the art to be suitable for use in infant formulas. Typical carbohydrate sources include sucrose, fructose, glucose, maltodextrin, lactose, corn syrup, corn syrup solids, rice syrup solids, rice starch, modified corn starch, modified tapioca starch, rice flour, soy flour, and the like.

The lipid source in the infant formula can be any lipid or fat known in the art to be suitable for use in infant formulas. Typical lipid sources include milk fat, safflower oil, egg yolk lipid, olive oil, coconut oil, palm oil, palm kernel oil, soybean oil, sunflower oil, fish oil and fractions derived thereof such as palm olein, medium chain triglycerides (MCT), and esters of fatty acids wherein the fatty acids are, for example, arachidonic acid, linoleic acid, palmitic acid, stearic acid, docosahexaenoic acid, eicosapentaenoic acid, linolenic acid, oleic acid, lauric acid, capric acid, caprylic acid, caproic acid, and the like. High oleic forms of various oils are also contemplated to be useful herein such as high oleic sunflower oil and high oleic safflower oil. Medium chain triglycerides contain higher concentrations of caprylic and capric acid than typically found in conventional oils, e.g., approximately three-fourths of the total fatty acid content is caprylic acid and one-fourth is capric acid.

Nutritionally complete compositions contain all vitamins and minerals understood to be essential in the daily diet and these should be present in nutritionally significant amounts. Those skilled in the art appreciate that minimum requirements have been established for certain vitamins and minerals that are known to be necessary for normal physiological function. Practitioners also understand that appropriate additional amounts (overages) of vitamin and mineral ingredients need to be provided to compensate for some loss during processing and storage of such compositions.

To select a specific vitamin or mineral compound to be used in the infant formula of the invention requires consideration of that compound's chemical nature regarding compatibility with the particular processing conditions used and shelf storage.

Examples of minerals, vitamins and other nutrients optionally present in the composition of the invention include vitamin A, vitamin B6, vitamin B12, vitamin E, vitamin K, vitamin C, folic acid, thiamine, inositol, riboflavin, niacin, biotin, pantothenic acid, choline, calcium, phosphorus, iodine, iron, magnesium, copper, zinc, manganese, chloride, potassium, sodium, selenium, chromium, molybdenum, taurine, and L-carnitine. Minerals are usually added in salt form. In addition to compatibility and stability considerations, the presence and amounts of specific minerals and other vitamins will vary somewhat depending on the intended infant population.

The infant formula of the invention also typically contains emulsifiers and stabilizers such as soy lecithin, carrageenan, and the like.

The infant formula of the invention may optionally contain other substances which may have a beneficial effect such as lactoferrin, nucleotides, nucleosides, immunoglobulins, and the like.

The infant formula of the invention is in concentrate liquid form, liquid ready to consume form, or powder form. If in powder form or concentrate liquid form, the formula is diluted to normal strength with water to be in a form ready to consume.

The osmolality of the liquid infant formula of the invention (when ready to consume) is typically about 100 to 1100 mOsm/kg H₂O, more typically about 200 to 700 mOsm/kg H₂O.

The infant formula of the invention can be sterilized, if desired, by techniques known in the art, for example, heat treatment such as autoclaving or retorting, and the like.

The infant formula of the invention can be packaged in any type of container known in the art to be used for storing nutritional products such as glass, lined paperboard, plastic, coated metal cans and the like.

The infant formula of the invention is shelf stable after reconstitution. By “shelf stable” is meant that the formula in a form ready to consume remains in a single homogenous phase (i.e., does not separate into more than one phase upon visual inspection) or that the thickener does not settle out as a sediment upon visual inspection after storage overnight in the refrigerator. With the thickened nature of the product, the formula of the invention also has the advantage of remaining fluid (i.e., does not gel into a solid mass when stored overnight in the refrigerator).

The invention provides a commercially acceptable product in terms of desired stability and physical characteristics and the product demonstrates little to no observable browning effect by-products associated with a Maillard reaction. Further, the inventive composition is substantially homogeneous for an acceptable period after reconstitution (or for the shelf-life if prepared as a liquid). The invention is particularly useful for infant formula preparations for the prevention and treatment of retinopathy of prematurity, although it is equally applicable to other elemental diets specific to a selected population that is at risk of, or is suspected of having, diabetic retinopathy, vascular proliferative retinopathy, or proliferation of abnormal vascularization, and the like.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### Example 1

This example illustrates the effect of exposure to Arg-gln dipeptide on the production of vascular endothelial cell growth factor (VEGF) in human retinal pigment epithelial cells (hRPE).

Human eyes were obtained from the National Disease Resource Interchange within 36 h of death. hRPE cells were prepared and maintained as described by Grant et al., in Curr. Eye Res., 9(4):323-335 (1990), and Enzmann et al., in Transpl Immunol, March 7(1): 9-14 (1999). For cell culture experiments, hRPE from passages 3-5 were used. All tissue culture media was purchased from Mediatech, Inc., Herndon, Fla. hRPE cultures were placed in glutamine-free medium for 24 hrs and then were exposed either to 0, 0.5, 1, 2.5 or 5 mg/ml of Arg-gln dipeptide for 48 hrs.

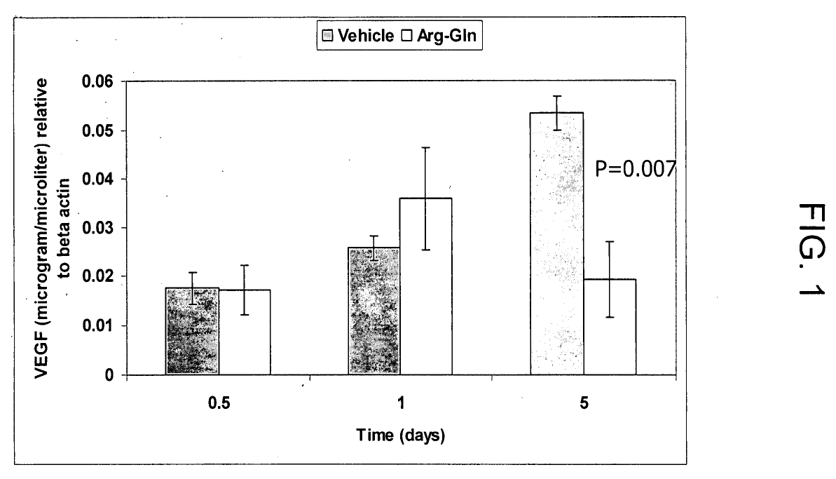

At the end of 48 hrs, VEGF content of the culture medium containing the hRPE cells was measured using the Quantikine® Human VEGF Immunoassay ELISA kit (R&D Systems, Minneapolis, Minn.). The results of the test are shown in FIG. 1. There, it is shown that exposure of the hRPE cells to the dipeptide resulted in a statistically significant decrease (reduced by 52.3±6.2% for 1.5 mM Arg-gln, P=0.002) in soluble VEGF expression into the culture medium. This showed that administration of the Arg-gln dipeptide was effective to reduce the VEGF production in human retinal cells.

### Example 2

This example illustrates the effect of parenteral administration of Arg-gln on VEGF mRNA production and abnormal retinal vascularization in a neonatal mouse model of retinopathy of prematurity.

Animals were treated in accordance with the ARVO “Statement for the Use of Animals in Ophthalmic and Vision Research.” Animal procedures were approved by the Institutional Animal Care and Use Committee of the University of Florida. C57BL6/J timed pregnant mice were obtained from Jackson Laboratories (Bar Harbor, Me.). The mice were housed in the University of Florida Health Science Center Animal Care facilities.

In the neonatal mouse model of oxygen-induced retinopathy, 7-day old mice were placed with their nursing dams in a 75% oxygen atmosphere for 5 days. (Smith et al., Invest Ophthalmol Vis Sci., 35(1):101-111 (1994). Mouse pups received twice daily intra-peritoneal injections (50 μl) starting on postnatal day 12 (P12) and continuing through postnatal day 17 (P17). In one experiment, injections included vehicle (0.9% sodium chloride) and the test compounds Ala-gly (5 g/kg/d), Arg-gln dipeptide (5 g/kg/d) and in a second experiment, different doses of Arg-gln (1.0, 2.5 and 5 g/kg/d) were tested.

After the fifth day following return to normoxia (P17), the animals were euthanized by injection of a lethal dose of a combination of ketamine (70 mg/kg body weight) and xylazine (15 mg/kg body weight) followed by cervical dislocation. The eyes were removed and fixed in 4% paraformaldehyde, embedded in paraffin, and sectioned as previously described. (Smith et al., Id.) Pre-retinal nuclei were counted by masked observers. Efficacy of treatment was calculated as the percent average nuclei per section in the eyes of Arg-Gln treated animals versus control animals.

For total RNA isolations from retina the animals were sacrificed and the eyes removed. The retina were then dissected away from the eye and stored in RNAlater® buffer (Ambion, Austin, Tex.) at 4° C. for subsequent isolation of protein or RNA.

VEGF ELISA

VEGF protein concentration was determined from isolated mouse retinas using the Quantikine® Human VEGF Immunoassay ELISA kit (R&D Systems, Minneapolis, Minn.). Pups were treated with the 5 g/kg/d dose of dipeptide and at varying time points after the pups were removed from oxygen on day 12. Eyes were enucleated at 0.5, 1 and 5 days post removal from high oxygen (n=14 eyes for each data point). Vehicle treated animals were compared to dipeptide-treated animals.

Real-Time RT-PCR

For VEGF mRNA measurements, total RNA was isolated from mouse retina using Trizol® reagent (Invitrogen, Carlsbad, Calif.) following the manufacture's protocol. The cDNA was synthesized using either 2 or 4 μg of total RNA and TaqMan® Reverse Transcription Reagents (PE Applied Biosystems) in 100 μL RT reaction. TaqMan® real time PCR analysis was applied using 1 μL cDNA per reaction and SYBR® Green PCR Core Reagents on ABI Prism Sequence Detection System 5700 (PE Applied Biosystems, Foster City, Calif. At the end of the PCR cycle, a dissociation curve was generated to ensure the amplification of a single product and the threshold cycle time (Ct values) for each gene was determined. Relative mRNA levels were calculated based on the Ct values and normalized to one of the following housekeeping genes: β-actin, cyclophilin, ribosomal protein S9 (100%).

Data Analysis

All data represent the mean±SEM. ANOVA was used to evaluate differences among groups, and individual contrasts were done using Student t-tests with Bonferroni correction was used to determine the significance between groups.

Results

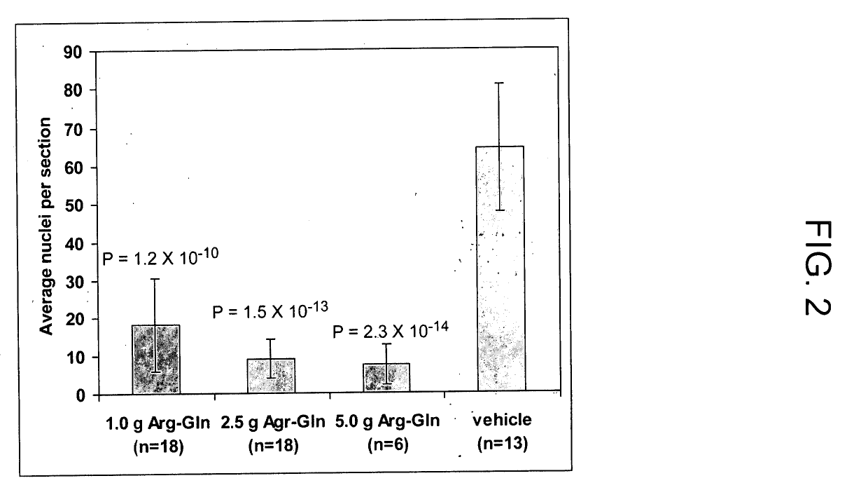

Five days of treatment of mouse pups with varying doses of Arg-gln (1.0, 2.5, and 5.0 g/kg/d) resulted in a significant reduction in preretinal nuclei. At the highest dose tested, there was approximately an 80% reduction. All treated samples showed significant decreases in preretinal nuclei (P<0.00001) (FIG. 2).

In FIG. 2, the dipeptide at the highest concentration was compared to the control dipeptide at the equivalent concentration. 5 g/kg/d resulted in an 82±7% reduction in preretinal vascularization (P<0.005 compared to vehicle) whereas the control dipeptide Ala-gly had no effect on the degree of pre-retinal neovascularization (2±5% reduction, P=0.45 compared to vehicle). Retinal flat mounts were prepared from Arg-gln-treated pups and compared to control dipeptide Ala-gly treated pups. The fluorescently labeled high molecular weight dextran delineated the vasculature and demonstrated that the retinal vessels from the Arg-gln treated pups have reduced neovascular tufts and vascular leakage compared to controls.

Effect of Arg-Gln Administration on VEGF mRNA in OIR Mice

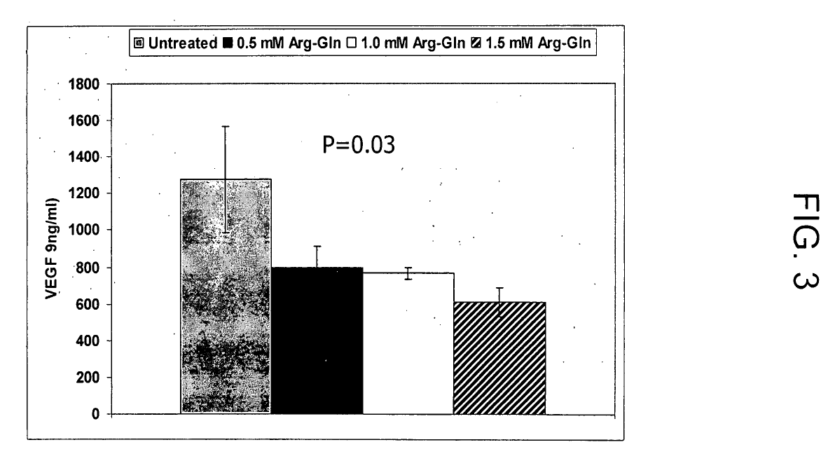

In the vehicle-treated animals (FIG. 3), there was a clear increase in the VEGF mRNA over the time period examined (P<0.001 compared to vehicle at day 0.5). At day 5 in the dipeptide-treated pups, there was a significant reduction in the VEGF mRNA (P=0.007 compared to vehicle treated at day 5).

Effectiveness for Related Conditions:

In the background section, several references are discussed that show the importance of vascular endothelial growth factor (VEGF) in the regulation and progression of retinopathy, and which show the involvement of glutamine in the upregulation of VEGF expression. In addition, Mino, et al., in the article Adenosine Receptor Antagonists and Retinal Neofascularization in Vivo, Investigative Ophthalmology & Visual Science, 42(13):3320-3324 (2001), state that vascular eye diseases, such as retinopathy of prematurity (ROP) and proliferative diabetic retinopathy (PDR) are characterized by abnormal growth of blood vessels across the retina. The authors state that although ROP and PDR differ in many respects, it is thought that neovascular growth arises in both diseases as a result of ischemic injury to retinal blood vessels. The authors relate how such injury occurs in premature infants to cause ROP, and how diabetes similarly leads to ischemic injury. They describe how ischemia initiates a series of events that lead to compensatory angiogenesis, and state that vascular endothelial growth factor is known to be a potential modulator for such activity. In conclusion they state their belief that the therapy studied (adenosine modulation) could provide a basis for developing pharmacologic therapies designed to prevent or treat the retinal neovascularization characteristic of proliferative retinopathies.

Commonalities in the pathology of retinopathies is also described in Raisler et al., Proc. Ntnl. Acad. of Sci., 99(13):8909-8914 (2002), where the authors state that pathologic neovascularization (NV) of the retina is central to several debilitating ocular diseases included proliferative diabetic retinopathy (PDR), age-related macular degeneration (AMD), and retinopathy of prematurity (ROP). Again, VEGF is identified as a key factor in the balance between endogenous positive growth factors (such as VEGF) and inhibitors of angiogenesis.

Articles by Afzal et al., Circulation Research, 93:500-506 (2003), Shaw et al., Investigative Ophthalmology & Visual Science, 44(9):4105-4113 (2003), and Grant et al., Expert Opin. Investig. Drugs, 3:1-19 (2004), and Ljubimov et al., Investigative Ophthalmology & Visual Science, 45(12):4583-4591 (2004), also discuss the common mechanisms present in the various types of diseases involving retinal angiogenesis and implicate the involvement of VEGF in the etiology of the diseases.

In a recent article by Pan et al., in Investigative Ophthalmology & Visual Science, 45(7):2413-2419 (2004), the authors reiterate the common facets of proliferative retinopathies that include retinopathy of prematurity (ROP), proliferative diabetic retinopathy (PDR) and “wet” age-related macular degeneration (ARMD), and state that “vascular endothelial growth factor is currently viewed as the major effector for retinal neovascularization in all proliferative retinopathies.”

In the present example, it has been shown that the administration of Arg-gln dipeptide significantly decreases the production of VEGF and VEGF mRNA in both human eye cells and in vivo in mice. Accordingly, it is maintained that those skilled in the art believe that the four retinopathies described in the present claims, namely: retinopathy of prematurity, diabetic retinopathy, vascular proliferative retinopathy, or proliferation of abnormal vascularization, share a number of common mechanisms. Also, they believe that a key step in the mechanisms involved VEGF. It is believed, therefore, that a therapy that involved modulation or regulation of VEGF, such as is demonstrated here, would be reasonably expected to have a similar affect in all retinopathies that share a mechanism involving VEGF.

### Example 3

This example illustrates the efficacy of enteral administration of arginyl-glutamine dipeptide for the prevention of retinopathy of prematurity in a mouse model of oxygen-induced retinopathy

All animals were treated in accordance with the ARVO “Statement for the Use of Animals in Ophthalmic and Vision Research.” Animal procedures have been approved by the Institutional Animal Care and Use Committee of the University of Florida.

C57BL6/J timed pregnant mice were obtained from Jackson Laboratories (Bar Harbor, Me.). The mice were housed in the University of Florida Health Science Center Animal Care facilities.

In the neonatal mouse model of oxygen-induced retinopathy, 7-day old mice were placed with their nursing dams in a 75% oxygen atmosphere for 5 days. Mouse pups received twice a day gavage feedings of dipeptide or control solution (50 microliter) starting on postnatal day 12 (P12) and continuing through postnatal day 17 (P17). Gavage feeds included vehicle (0.9% sodium chloride) and the test compounds and different doses of Arg-Gln (1.0, 2.5 and 5 gm/kg/d). The daily dosage of the dipeptide was divided evenly between the two daily gavage feedings.

After the fifth day following return to normoxia (P17), the animals were euthanized by injection of a lethal dose of a combination of ketamine (70 mg/kg body weight) and xylazine (15 mg/kg body weight) followed by cervical dislocation. The eyes were removed and fixed in 4% paraformaldehyde, embedded in paraffin, and sectioned. Pre-retinal nuclei were counted by masked observers. Efficacy of treatment was calculated as the percent average nuclei per section in the eyes of Arg-Gln treated animals versus control animals.

For total RNA isolations from retina the animals were sacrificed and the eyes removed. The retina was then dissected away from the eye and stored in RNAlater® buffer (Ambion, Austin, Tex.) at 4° C. for subsequent isolation of protein or RNA.

Some of the eyes were taken for qualitative retinal flatmount analysis. For these mice, at the time of euthanasia, the mice were perfused with FITC-labeled dextran to visualize the vasculature. The eyes were enucleated and incubated in 4% formaldehyde and then in PBS. The neural retina was dissected from the RPE-choroid-sclera complex and flatmounted with four to seven radial cuts and examined and photographed separately using confocal microscopy (MRC-1024 Confocal Laser Scanning System, Bio-Rad, Hercules, Calif.).

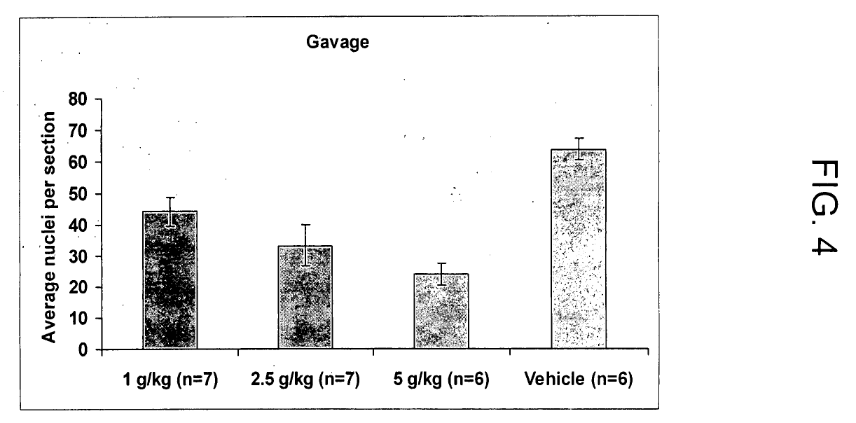

Results of enteral administration of the Arg-gln dipeptide are shown in FIG. 4, which is a bar chart showing the average nuclei per section in the eyes of neonatal mice exposed to hyperoxia as a function of the dosage of Arg-gln dipeptide, with dosage levels of zero (vehicle), 1 gm/kg·day, 2.5 gm/kg·day, and 5 gm/kg·day. The greater number of nuclei corresponds to greater retinal vascular proliferation and therefore retinopathy. Therefore, a lower number of nuclei indicates a more favorable outcome. In FIG. 4, it is seen that the average number of nuclei per section was decreased at all levels of administration of the dipeptide as compared with subjects receiving only vehicle. A recognizable dose/response was shown for dosages of the dipeptide between 1 gm/kg·day and 5 gm/kg·day, and the highest level of effectiveness was shown at 5 gm/kg·day.

Rationale for Dosages:

The overall purpose of the experiments in animals is to establish safety, appropriate dosage range and efficacy prior to evaluation in human neonates at risk for retinopathy of prematurity. Since the ultimate purpose of the Arg-gln dipeptide is to provide a safe and easily absorbable preparation that can be used to provide appropriate nutritional intakes of arginine and glutamine in human infants who might not be receiving appropriate quantities of these amino acids in their diets, the dosages in the animal studies are modeled after human premature neonate recommended intakes.

In addition, several studies now exist wherein glutamine or arginine have been administered to human neonates at dosages that can now be presumed as safe, although the efficacy of such treatments is in doubt. For example, Roig et al., in J. Pediatr., 131(5):691-699 (1997), reported enteral glutamine supplementation in low birth weight infants to be useful, while Poindexter et al., in Pediatrics, 113:1209-1215 (2004) report that glutamine supplementation is not useful for reducing mortality or late-onset sepsis in the same population. Robert et al., in Pediatric Res., 51(1):87-93 (2002), reported that IV administration of glutamine at 500 mg/kg·day failed to enhance rates of protein synthesis, but may have acute protein-sparing effects. Further reports of glutamine supplementation appear in Mercier et al., Clinical Nutrition, 22(2):133-137 (2003) (700 mg/kg·day fed enterally), Parimi et al., Am. J. Clin. Nutr., 79:402-409 (2004) (600 mg/kg·day fed enterally), van den Berg et al., Am J. Clin Nutr, 81:1397-1404 (2005) (300 mg/kg·day fed enterally), and Vaughn et al., J. of Pediatrics, pp. 662-668 June (2003) (300 mg/kg·day fed enterally).

In Amin et al., J. of Pediatrics, 140(4):425-431 (2002), arginine supplementation by both oral and parenteral means at about 260 mg/kg·day was found to prevent necrotizing enterocolitis in premature infants.

Human premature infants' protein requirements are between 34 grams/kg/day. Approximately 10% of human milk and formula protein is glutamine, therefore the intake of glutamine would be 0.3-0.4 grams/kg/d. Several studies of enteral and intravenous glutamine have been done and the dosages have ranged from 0.3 grams/kg/d to slightly greater than 0.6 grams/kg/d. One study of arginine supplementation used to prevent necrotizing enterocoltis utilized a daily supplement of 1.5 mmole/kg/d (IV or enterally), which is approximately 0.26 grams/kg/d. Further information can be found in Dietary Reference Intakes for Energy, Carbohydrate, Fiber, Fat, Fatty Acids, Cholesterol, Protein, and Amino Acids (Macronutrients), Publ., Food and Nutrition Board (FNB) (2005), Thureen et al., Pediatric Research, 57(5):95R-98R (2005), and Räihä et al., J. Pediatric Gastroenterology and Nutrition, 35:275-281 (2002).

Infant rodents' protein requirements are about 10 times that of the human premature, hence they receive approximately 30-40 grams/kg/d protein from their mothers' milk. See, Reeds et al., Proc. Of the Nutrition Soc., 59:87-97 (2000). With 10% of this being glutamine, they receive approximate 3-4 grams/kg/d of glutamine. Hence the dosage range for the mouse studies (0, 1, 2.5 and 5 grams/kg/d of the dipeptide) is based primarily on the presumed glutamine intakes of rodents, which in turn is based on their protein intakes. Dosage ranges that would be useful in human neonates would be expected to be roughly 1/10th that of dosage rates found to be efficacious in mice. By way of example, to obtain in human neonates a response equivalent to that exhibited in rodents at a dosage of the dipeptide of about 5 gm/kg·day, one would expect to administer about 0.5 gm/kg·day of the dipeptide to the human neonate. Due to the protein nutritional requirements of human neonates, one would likely employ dosages not much greater than 1 gm/kg/d.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions by those of ordinary skill in the art without departing from the scope of the invention, it is intended that all matter contained in the above description and shown in the accompanying drawings shall be interpreted as illustrative and not in a limiting sense. In addition it should be understood that aspects of the various embodiments may be interchanged both in whole or in part.

What is claimed is:

1. A method for preventing and/or treating abnormal vascular proliferation in a subject, the method comprising enterally administering arginine and glutamine to the subject in about equimolar amounts to provide a total amount of arginine and glutamine that is effective to prevent or treat the abnormal vascular proliferation.

2. The method according to claim 1, wherein administering arginine and glutamine comprises administering Arg-gln dipeptide.

3. The method according to claim 1, wherein the total amount of arginine and glutamine is from about 100 mg/kg·day to about 1000 mg/kg·day.

4. The method according to claim 1, wherein the total amount of arginine and glutamine is from about 200 mg/kg·day to about 800 mg/kg·day.

5. The method according to claim 1, wherein the total amount of arginine and glutamine is from about 250 mg/kg·day to about 600 mg/kg·day.

6. The method according to claim 1, wherein the total amount of arginine and glutamine is from about 300 mg/kg·day to about 600 mg/kg·day.

7. The method according to claim 1, wherein the total amount of arginine and glutamine is from about 300 mg/kg·day to about 500 mg/kg·day.

8. The method according to claim 1, wherein the total amount of arginine and glutamine is about 500 mg/kg·day.

9. The method according to claim 1, wherein the subject is a human infant that is in need of prevention or treatment of abnormal vascular proliferation.

10. The method according to claim 9, wherein the human infant is in need of prevention or treatment of retinopathy of prematurity, or vascular proliferative retinopathy.

11. The method according to claim 9, wherein the human infant is a newborn infant.

12. The method according to claim 9, wherein the human infant is a premature infant.

13. The method according to claim 2, wherein the subject is a human infant and enterally administering Arg-gln dipeptide comprises administering liquid infant formula containing Arg-gln dipeptide to the infant.

14. The method according to claim 13, wherein the liquid infant formula is nutritionally complete.

15. The method according to claim 2, wherein enterally administering Arg-gln dipeptide comprises administering a nutritional supplement containing Arg-gln dipeptide to the subject.

16. An infant formula comprising arginine and glutamine in about equimolar amounts and in a total amount to provide the combination of arginine and glutamine to a human infant in an amount of from about 100 mg/kg·day to about 1000 mg/kg·day.

17. The infant formula according to claim 16, wherein the formula is a liquid in which the concentration of the combination of arginine and glutamine is from about 0.4 gm/liter to about 5 gm/liter.

18. The infant formula according to claim 17, wherein the concentration of the combination of arginine and glutamine is from about 1 gm/liter to about 4 gm/liter.

19. The infant formula according to claim 17, wherein the concentration of the combination of arginine and glutamine is from about 2 gm/liter to about 3 gm/liter.

20. The infant formula according to claim 16, wherein the arginine and glutamine are provided in whole or in part by arginyl-glutamine dipeptide, or a salt or precursor thereof.

21. The infant formula according to claim 16 wherein the infant formula comprises arginyl-glutamine dipeptide, or a salt or precursor thereof, in an amount sufficient for the prevention or treatment of a condition that is selected from retinopathy of prematurity, diabetic retinopathy, vascular proliferative retinopathy, or proliferation of abnormal vascularization.

22. The infant formula according to claim 16, wherein the infant formula is nutritionally complete.

23. The infant formula according to claim 16, wherein the arginine and glutamine consist essentially of arginyl-glutamine dipeptide.

24. An infant nutritional supplement comprising amino acids consisting essentially of arginine and glutamine in about equimolar amounts.

25. The infant nutritional supplement according to claim 24, wherein the arginine and glutamine are provided by Arg-gln dipeptide.

26. The infant nutritional supplement according to claim 24, wherein arginine and glutamine are provided in equimolar amounts.
